(19) United States
(12) Patent Application Publication
(10) Pub. No.: US 2021/0030764 A1
(43) Pub. Date: Feb. 4, 2021

(54) METHOD OF TREATING CANCER

(71) Applicant: University of Louisiana at Lafayette, Lafayette, LA (US)

(72) Inventor: Radhey Srivastava, Youngsville, LA (US)

(21) Appl. No.: 16/943,323

(22) Filed: Jul. 30, 2020

Related U.S. Application Data
(60) Provisional application No. 62/880,726, filed on Jul. 31, 2019.

(51) Int. Cl. A61K 31/555

(52) U.S. Cl. CPC A61K 31/555

(57) ABSTRACT

Disclosed herein is a method of treating patients with NSCLC subtypes of cancer by reducing tumor burden, comprising administering at least one dose of purified Ruthenium (III) Pyrazole complexes to patients suffering from NSCLC, wherein said dose is therapeutically sufficient. In one or more embodiments, the Ruthenium III Pyrazole is RuCl₃(DMSO)(dmPyz)₂.

### CROSS REFERENCE TO RELATED APPLICATIONS

This application claims benefit to the U.S. Provisional Patent Application No. 62/880,726, entitled “METHOD OF TREATING CANCER”, filed Jul. 31, 2019.

### STATEMENT REGARDING FEDERALLY SPONSORED RESEARCH OR DEVELOPMENT

Not Applicable.

### REFERENCE TO A “SEQUENCE LISTING,” A TABLE, OR A COMPUTER PROGRAM

Not Applicable.

### DESCRIPTION OF THE DRAWINGS

FIG. 1 is an Ortep diagram of mer-[RuCl3(DMSO)(dmpyz)2.

FIG. 2 is the structure of a pyrazole.

FIG. 3 is a table showing test results for Ruthenium complexes as anticancer agent.

FIG. 4 is an Ortep diagram of mer-[RuCl₃(DMSO)(pyz)₂].

FIG. 5 is an Ortep diagram of mer-[RuCl₃(bpy)(dmpyz)₂].

FIG. 6 is an Ortep diagram of mer-[RuCl₃(DMSO)₂(pyz)].

### BACKGROUND OF THE INVENTION

There have been numerous advancements in the treatment of cancer, leading to the availability of many cancer therapies in the market. Great success has been found through platinum derived anti-cancer drugs. For example, Cisplatin, a chemotherapy medication, is one of the most widely used drugs in clinical use and has shown great promise. Cisplatin is a platinum (Pt) containing chemotherapeutic and considered an essential medicine by the WHO. Cisplatin's clinical success spawned decades-long interest in leveraging transition metals and associated compounds for treatment of human malignancies. However, the effectiveness of this drug, like many of its counterparts, is limited for a variety of reasons. First and foremost, the drug is toxic to human cells and has the propensity to cause severe, unwanted side effects. Additionally, studies have noticed a growing resistance to the drug from some forms of cancer.

While great strides have been made, the field leaves room for additional advancement. The American Cancer Society estimates that over 1,762,000 new cancer cases will be diagnosed in the United States in 2019 alone, and that cancer will cause over 600,000 deaths. Any new cancer treatment is welcomed in the industry as a breakthrough in medical science. But the industry and scientific community are constantly striving to find complexes of transition metals with wider ranges of activities while causing lower systematic toxicities. Any new anti-cancer treatment that also reduces the unwanted side effects and toxicity would be a windfall.

Ruthenium (Ru) based compounds have received interest in recent years, due in part to their improved ability to specifically target cancerous tissue while sparing healthy tissue. Ru compounds are known to impart their anticancer effect largely through genotoxic (e.g, DNA damage, topoisomerase inhibition, etc) activity. This can be problematic as it is often difficult to fully localize treatment to cancerous cells only. Thus, the community is attempting to develop complexes that can more specifically target cancer subtypes, further reducing undesirable damage to healthy cells. One such treatment is disclosed herein that employs Ruthenium (III) complexes.

### BRIEF SUMMARY OF THE INVENTION

Disclosed herein is a method of treating cancer through the proper dosing of a neutral ruthenium (III)-pyrazole complex. More specifically, the invention relates to the treatment of certain types of cancer with dosage of complexes of ruthentium (III) reacted with pyrazoles to as to obtain the desired complex.

In one or more embodiments, the complex is a Ruthenium pyrazole selected from the group comprising [RuCl₃(DMSO-S)(pyz)₂], [RuCl₃(DMSO-S) (DMSO-O)(pyz)], [RuCl₃(bpy)(dm pyz)], and [RuCl₃(DMSO-S)(dm pyz)₂]. In a preferred embodiment, the complex is RuCl₃(DMSO)(dmPyz)₂. As used herein, pyz is pyrazole; DMSO is Dimethylsulfoxide, dmpyz is 3,5-dimethylpyrazole, and bpy is 2,2′-bipyridine.

### DETAILED DESCRIPTION

The subject matter of the present invention is described with specificity herein to meet statutory requirements. However, the description itself is not intended to necessarily limit the scope of claims. Rather, the claimed subject matter might be embodied in other ways to include different steps or combinations of steps similar to the ones described in this document, in conjunction with other present or future technologies.

Furthermore, the described features, structures, or characteristics may be combined in any suitable manner in one or more embodiments. In the following description, numerous specific details are provided, such as examples of dosages, dosage means, amounts of dosage(s), and application times

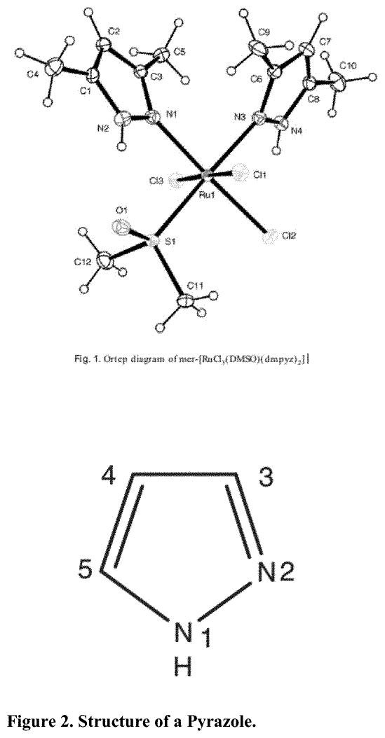

Turning first to the complex itself, FIG. 1 shows the Ortep diagram of a particular Ruthenium (III) pyrazole complex, mer-RuCl₃(DMSO)(dmPyz)₂. As more fully explained in Synthesis, characterization, and anticancer activity of rutheneium-pyrazole complexes, Journal of Inorganic Biochemistry 111 (2012) 33-39, which is incorporated herein by reference, this complex may crystallizes in the monoclinic space group Cc that adopts a distorted octahedral geometry with meridional arrangement. Both pyrazole ligands are coordinated to ruthenium through the tertiary ring nitrogen atom N2 as depicted in FIG. 2, The average Ru—Cl bond length, 2.344(9) Å is comparable to other related Ru(III) complexes. The Ru—S bond distance, 2.2783 is appreciably shorter than the average value of 2.34(1) Å found for the Ru(III)-S compounds trans to DMSO-S in the precursor. Likewise the average bond length of Ru—N, 2.089 Å is comparable to Ru(III)-N(imidazole), 2.079 Å. The bond angles of N1Ru1S1, 178.57(11)°; and Cl2Ru1N3, 176.78(9)° are slightly smaller than the known values of octahedral complexes of ruthenium (III).

Synthesis. The complex may be formed by the displacement of two DMSO ligands from the precursor, mer-[RuCl₃(DMSO-S)₂(DMSO-O) with pyrazole. A solution of mer-[RuCl₃(DMSO-S)₂(DMSO-O)] (0.2 g, 0.45 mmol) and 3,5-dimethylpyrazole (0.334 g, 3.48 mmol) in dichloromethane (15 mL) was heated to reflux for 9 hours. The brown solution was evaporated to 3 mL and then 1 mL of hexane was added. On cooling at 4° C., a reddish-orange solid was separated (yield 0.262 mmol, 58%). The product was purified by column chromatography with ethyl acetate and hexane (40/60) as eluent. Anal. Calcd. C12H20Cl₃N₄ORuS.H₂O (495.83): C, 29.06; H, 4.95, N, 11.35. Found: C, 29.18; H, 5.24; N, 10.95. Selected IR absorption bands in KBr (cm-1): ν SO1086 (S-DMSO), ν C═C (pyz) 1632, ν C═N (pyz) 1398. UV-vis (H₂O): 362 nm (ε=12.20 M-1 cm-1).

RuCl₃(DMSO)(dmPyz)₂ was crystallized in the monoclinic space group P2₁/n. The X-ray structure in FIG. 2 shows a distorted octahedral geometry (FIG. 1) with three chlorine atoms in a mer configuration. The average Ru—Cl bond length, 2.3411(9). The Ru—S bond distance, 2.3050(9) Å is shorter than the average value of 2.34(1) Å found for the Ru(III)-S compounds trans to DMSO-S in the precursor. The average bond length of Ru—N, 2.1005(3) Å, is longer than Ru(III)-N(imidazole), 2.079 Å, and the average bond length (Ru—N) of 1 (2.089 Å). The bond angles of N1Ru1S1, 176.62(9)° and N1RuCl2, 176.83(3)° are close to the known values of octahedral complexes of ruthenium (III).

TABLE 1
bond length and bond angle

| | | | |
|---|---|---|---|
| Ru1-N1 | 2.095(3) | N1-Ru1-N3 | 88.81(12) |
| Ru1-N3 | 2.106(3) | N1-Ru1-S1 | 92.28(9) |
| Ru1-S1 | 2.3050(9) | N3-Ru1-S1 | 176.62(9) |
| Ru1-Cl1 | 2.3356(9) | N1-Ru1-Cl1 | 91.14(9) |
| Ru1-Cl2 | 2.3466(9) | N3-Ru1-Cl1 | 91.36(9) |
| Ru1-Cl3 | 2.3411(9) | N3-Ru1-Cl3 | 90.11(9) |
| | | Cl3-Ru1-Cl1 | 178.38(3) |
| | | N1-Ru1-Cl2 | 176.83(9) |
| | | Cl3-Ru1-Cl2 | 89.24(3) |
| | | Cl1-Ru1-Cl2 | 90.13(3) |

As discussed in the Journal of Inorganic Biochemistry 2012 article supra, it was previously discovered that certain Ru-Pyrazole complexes exhibited specific antibiological activity in the MCF7 (ER⁺PR⁺) breast cancer cells. However, it was not shown, or even envisioned, that the currently discussed Ruthenium (III) complex would exhibit any anticancer attributes in subtypes of non-small cell lung cancer (NSCLC). It must be noted that RuCl₃(DMSO)(dmPyz)₂ does not exhibit general lethality and, therefore, would not ordinarily be believe to be an anti-cancer agent.

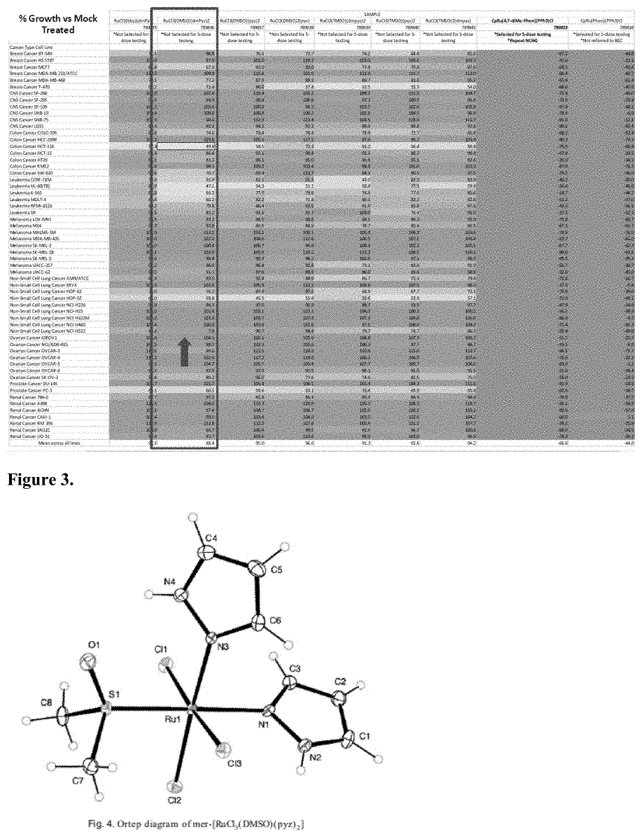

Still, recent tests show promise that dosage of RuCl₃(DMSO)(dmPyz)₂ is effective against NSCLC subtypes. FIG. 3 is a table showing the results of Ruthenium complex screening conducted to test the efficacy of the current Ruthenium (III) complexes to various cancer subtypes. The test found 97% inhibition of NSCLC with a treatment of 10 μM purified. This suggests that the IC₅₀ for the complex in the H522 cell lines is under 10 μM, allowing smaller doses to be effective.

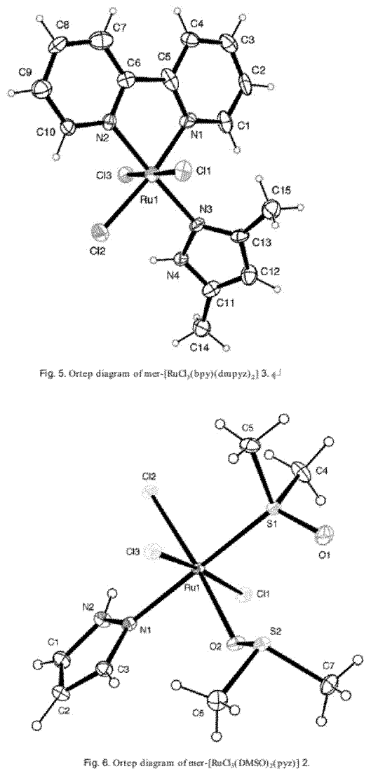

It is also envisioned that other Ruthenium complexes may be viable anticancer agents. Representative examples of such complexes are shown in FIGS. 4-6 and include [RuCl₃(DMSO-S)(pyz)₂], [RuCl₃(DMSO-S) (DMSO-O)(pyz)], and [RuCl₃(bpy)(dm pyz)].

Reference throughout this specification to features, advantages, or similar language does not imply that all of the features and advantages that may be realized should be or are in any single embodiment. Rather, language referring to the features and advantages is understood to mean that a specific feature, advantage, or characteristic described in connection with an embodiment is included in at least one embodiment. Thus, discussion of the features and advantages, and similar language, throughout this specification may, but do not necessarily, refer to the same embodiment.

Reference throughout this specification to “one embodiment,” “an embodiment,” or similar language means that a particular feature, structure, or characteristic described in connection with the embodiment is included in at least one embodiment. Thus, appearances of the phrases “in one embodiment,” “in an embodiment,” and similar language throughout this specification may, but do not necessarily, all refer to the same embodiment.

It should be understood that the drawings are not necessarily to scale; instead, emphasis has been placed upon illustrating the principles of the invention. In addition, in the embodiments depicted herein, like reference numerals in the various drawings refer to identical or near identical structural elements.

Moreover, the terms “substantially” or “approximately” as used herein may be applied to modify any quantitative representation that could permissibly vary without resulting in a change to the basic function to which it is related.

1. A method of treating cancer patients by reducing tumor burden, comprising the steps of:
a. Administering at least one dose of a Ruthenium (III) complex to the cancer patient, wherein said dose is therapeutically sufficient to treat a cancer cell.

2. The method of claim 1 wherein the dose is purified prior to administering.

3. The method of claim 1 wherein the cancer cell is a non-small cell lung cancer subtype.

4. The method of claim 1 wherein the Ruthenium (III) complex is formed by the displacement of two DMSO ligands from a precursor, mer-[RuCl₃(DMSO-S)₂(DMSO-O), with pyrazole.

5. The Method of claim 1 wherein the Ruthenium (III) complex is a Ruthenium (III) pyrazole complex.

6. The method of claim 2 wherein the dose is purified by column chromatography.

7. The Method of claim 3 wherein the Ruthenium (III) complex is elected from the group comprising [RuCl₃(DMSO-S)(pyz)₂], [RuCl₃(DMSO-S) (DMSO-O)(pyz)], [RuCl₃(bpy)(dm pyz)], and [RuCl₃(DMSO-S)(dm pyz)₂].

8. The method of claim 3 wherein the Ruthenium (III) complex is RuCl₃(DMSO)(dmPyz)₂.

9. The method of claim 8 wherein the dose comprises a purified RuCl₃(DMSO)(dmPyz)₂.

10. The method of claim 9 wherein the at least one dose comprises between 0.1 and 10 μM of purified RuCl₃(DMSO)(dmPyz)₂.

11. The method of claim 10 wherein a second dose is administered days after the first dose.

12. The method of claim 10 wherein at least five doses are administered.

13. The method of claim 10 wherein each dose is administered on different days.
